(19) United States
(12) Patent Application Publication
(10) Pub. No.: US 2011/0280914 A1
(43) Pub. Date: Nov. 17, 2011

(54) HYDROGELS CROSSLINKED WITH GOLD NANOPARTICLES AND METHODS OF MAKING AND USING THEREOF

(75) Inventors: Glenn D. Prestwich, Eastbound, WA (US); Aleksander Skardel, Salt Lake City, UT (US); Jianxing Zhang, Salt Lake City, UT (US)

(73) Assignee: University of Utah Research Foundation, Salt Lake City, UT (US)

(21) Appl. No.: 13/146,564

(22) PCT Filed: Dec. 17, 2009
(86) PCT No.: PCT/US09/68470

§ 371 Date: Jul. 27, 2011

Related U.S. Application Data
(60) Provisional application No. 61/148,526, filed on Jan. 30, 2009.

(51) Int. Cl. A61K 9/00; C07K 14/435; A61K 47/36; A61K 47/38; A61K 47/42; A61K 35/12; A61K 35/30; A61K 35/34; A61K 35/407; A61K 35/36; A61K 35/14; A61K 35/39; C12N 5/071; C12N 5/077; C12N 5/0793; C12N 5/078; C12Q 1/02; C12P 21/08; C12P 21/00; C07H 23/00; B82Y 5/00

(52) U.S. Cl. 424/400; 536/121; 536/54; 536/20; 536/3; 536/2; 536/21; 536/53; 536/43; 530/354; 514/777; 514/781; 514/779; 514/774; 424/93.7; 435/382; 435/395; 435/29; 435/70.3; 435/325; 977/773; 977/810; 977/896; 977/923; 977/906

(57) ABSTRACT

Described herein are composites useful in tissue and organ engineering. In one aspect, the composite comprises the reaction product between a macromolecule comprising at least one thiol group and a gold nanoparticle. The thiolated macro-molecule crosslinks with the gold nanoparticle to produce a composite that is useful in anchoring cells. The composites can be used to form multi-layer 3-D structures, where the cells in each layer can aggregate and fuse with one another to form tissues and organs.

### CROSS-REFERENCE TO RELATED APPLICATIONS

This application claims benefit of U.S. Provisional Application Ser. No. 61/148,526 filed on Jan. 30, 2009, which is hereby incorporated by reference in its entirety for all of its teachings.

### ACKNOWLEDGEMENTS

The research leading to this invention was funded in part by an National Science Foundation Frontiers in Integrative Biological Research (NSF FIBR) Grant No. EF-0526854. The U.S. Government has certain rights in this invention.

### BACKGROUND

As the world's population and average human lifespans increase, global medical needs will also increase. These needs include, but are not limited to, organs and tissues for implantation and assessing safety and efficacy pharmaceutical treatments. There is already a massive shortage of donor organs, and while preclinical testing of candidate drugs in animals is well established, it is neither particularly efficient nor predictive of the clinical outcome. For years now, it has been proposed that tissue engineering would offer one solution; however, the production of viable and functional organs for many complex metabolic tissues remains an elusive goal.

Complexity of cell and tissue organization within an organ has proven to be one of those roadblocks. Simply injecting or implanting masses of cells in vivo or growing cells in vitro does not yield an organ. Instead, another method of organizing cells into the proper three-dimensional construct to facilitate tissue formation is needed. Bioprinting is one such method; it involves placing encapsulated cells or cell aggregates into a 3-D construct using a 3-axis analogue of an inkjet printer. This device has the ability to print cell aggregates, sECM hydrogels, and cell-seeded microspheres (i.e., the “bioink”), as well as cell-free polymers that provide structure (i.e., the “biopaper”). A computer-assisted design can be used to guide the placement of specific types of cells and polymer into precise geometries that mimic actual tissue and/or organ construction. With the appropriate cell types already in the appropriate positions, the organ can then be allowed to mature and gain full functionality in an appropriate bioreactor or in vivo environment.

To date, a complete organ has not been printed; however, cell aggregates and cell sausages have been printed layer-by-layer into tubular formations within agarose, showing the feasibility or printing vessels and other tubular structures. After printing, the property of tissue liquidity allowed the aggregates and sausages to fuse into a singular seamless structure.

Printing with agarose has intrinsic limitations. Despite being bio-inert, and thus safe for work with cells, agarose does not support cell adhesion, it requires high temperatures, and it is not biodegradable in mammalian systems. Moreover, agarose gels can only be printed by preforming the gel into a tubular shape with the same diameter of the printing devices, and it cannot be easily removed from the printed construct. Thus, it can only be used as a permanent structural component, limiting its potential use in bioprinting, as well as limiting the advancement of bioprinting itself.

### SUMMARY

Described herein are composites useful in tissue and organ engineering. In one aspect, the composite comprises the reaction product between a macromolecule comprising at least one thiol group and a gold nanoparticle. The thiolated macromolecule crosslinks with the gold nanoparticle to produce a composite that is useful in anchoring cells. The composites can be used to form multi-layer 3-D structures, where the cells in each layer can aggregate and fuse with one another to form tissues and organs. Also described herein are methods for making and using the composites. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate several aspects described below. The terms Extracel, Glycosil, and Gelin-S used in the drawings and elsewhere are trademarked products of Glycosan BioSystems, Inc.

FIG. 1 is a diagram showing the crosslinking strategy used to prepare the composites described herein. FIG. 1(a) shows the crosslinking strategy for a hydrogel having a thiolated macromolecule that forms bonds with the surface of the gold nanoparticle (AuNP) creating a network. FIG. 1(b) is a diagram showing the crosslinking strategy used to prepare a hydrogel having a thiolated macromolecule that is capable of forming bonds with both gelatin-DTPH and the surface of the gold nanoparticle (AuNP).

FIG. 2 shows: (A) a scheme illustrating layer by layer printing; (B)a single hand-printed hydrogel tube; (C) a 10 layer-high pyramid; and (D) a square base useful in applying the composites described herein; and (E) a 5 layered square structure composed of five layers of composite.

FIG. 3 shows an example of a printed pattern of hydrogel composite described herein.

FIG. 4 shows (A) the nScrypt bioprinter useful herein and (B) a line of composite (Extracel-AuNP hydrogel) being printed from the bioprinter's printer head.

FIG. 5 shows the percent of (A) murine NIH 3T3 fibroblasts, (B) human HepG2 C3A cells, and (C) human Intestine 407 cells that were viable on days 3 and 7 of culture on a composite described herein (Extracel-AuNP hydrogel) and control (Extracel hydrogel).

FIG. 6 shows rheology data from various hydrogel formulations. Stiffness increased with increased gold nanoparticle size, CMHA-S (Glycosil) concentration and percentage, and crosslinking time.

FIG. 7(a) shows a schematic depicting the approach for printing a cellularized construct with the AuNP hydrogel. Layers of cellularized hydrogel circles surrounded by cell-free hydrogel are printed on top of one another, building a tubular structure upwards, maturing into tissue during time in culture. FIG. 7(b) shows the Fab@Home Model 1 2-Syringe printing system which can be used in bioprinting. FIG. 7(c) shows a tubular tissue construct printed without the central core and outer support hydrogel for visualization. FIG. 7(d) shows a complete tubular tissue construct. The cell-containing portion forming the tube was supplemented with HA-Bodipy for fluorescent visualization under 365 nm UV light.

FIG. 8(a) shows a gross light microscopy image of a printed construct after 4 weeks of culture showing increased opacity. FIG. 8(b) Masson Trichrome stain of a tissue construct. Visible are numerous cell nuclei (black) and collagen matrix (blue). FIG. 8(c) shows a trichrome stain of a negative control hydrogel, containing gelatin-DTPH, but no cells. The lack of blue stain for collagen indicates that the presence of gelatin does not result in a false positive stain for collagen. FIG. 8(d) shows cells stained with IHC stain for procollagen. Brown staining, as shown by arrows, indicates active production of endogenous collagen. FIG. 8(e) shows a skin tissue positive control sample illustrating the specificity of the stain.

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a macromolecule” includes mixtures of two or more such macromolecules, and the like.

“Optional” or “optionally” means that the subsequently described event or circumstance can or cannot occur, and that the description includes instances where the event or circumstance occurs and instances where it does not. For example, the phrase “optionally substituted lower alkyl” means that the lower alkyl group can or can not be substituted and that the description includes both unsubstituted lower alkyl and lower alkyl where there is substitution.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included.

A residue of a chemical species, as used in the specification and concluding claims, refers to the moiety that is the resulting product of the chemical species in a particular reaction scheme or subsequent formulation or chemical product, regardless of whether the moiety is actually obtained from the chemical species. For example, hyaluronan that contains at least one —OH group can be represented by the formula Y—OH, where Y is the remainder (i.e., residue) of the hyaluronan molecule.

The term “alkyl group” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is an alkyl group containing from one to six carbon atoms.

The term “polyalkylene group” as used herein is a group having two or more CH₂ groups linked to one another. The polyalkylene group can be represented by the formula —(CH₂)ₙ—, where n is an integer of from 2 to 25.

The term “polyether group” as used herein is a group having the formula —[(CHR)ₙO]ₘ—, where R is hydrogen or a lower alkyl group, n is an integer of from 1 to 20, and m is an integer of from 1 to 100. Examples of polyether groups include, polyethylene oxide, polypropylene oxide, and polybutylene oxide.

The term “polythioether group” as used herein is a group having the formula —[(CHR)ₙS]ₘ—, where R is hydrogen or a lower alkyl group, n is an integer of from 1 to 20, and m is an integer of from 1 to 100.

The term “polyimino group” as used herein is a group having the formula —[(CHR)ₙNR]ₘ—, where each R is, independently, hydrogen or a lower alkyl group, n is an integer of from 1 to 20, and m is an integer of from 1 to 100.

The term “polyester group” as used herein is a group that is produced by the reaction between a compound having at least two carboxylic acid groups with a compound having at least two hydroxyl groups.

The term “polyamide group” as used herein is a group that is produced by the reaction between a compound having at least two carboxylic acid groups with a compound having at least two unsubstituted or monosubstituted amino groups.

The term “aryl group” as used herein is any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorus. The aryl group can be substituted or unsubstituted. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “hydrocarbyl group” as used herein means the monovalent moiety obtained upon removal of a hydrogen atom from a parent hydrocarbon. Representative of hydrocarbyl are alkyl of 1 to 20 carbon atoms, inclusive, such as methyl, ethyl, propyl, butyl, pentyl, hexyl, heptyl, octyl, nonyl, undecyl, decyl, dodecyl, octadecyl, nonodecyl, eicosyl, heneicosyl, docosyl, tricosyl, tetracosyl, pentacosyl and the isomeric forms thereof; aryl of 6 to 12 carbon atoms, inclusive, such as phenyl, tolyl, xylyl, naphthyl, biphenyl, tetraphenyl and the like; aralkyl of 7 to 12 carbon atoms, inclusive, such as benzyl, phenethyl, phenpropyl, phenbutyl, phenhexyl, napthoctyl and the like; cycloalkyl of 3 to 8 carbon atoms, inclusive, such as cyclopropyl, cyclobutyl, cyclopentyl, cyclohexyl, cycloheptyl, cyclooctyl and the like; alkenyl of 2 to 10 carbon atoms, inclusive, such as vinyl, allyl, butenyl, pentenyl, hexenyl, octenyl, nonenyl, decenyl, undececyl, dodecenyl, tridecenyl, pentadecenyl, octadecenyl, pentacosynyl and isomeric forms thereof. Preferably, the hydrocarbyl group has 1 to 20 carbon atoms, inclusive.

The term “substituted hydrocarbyl and heterocarbyl” as used herein means the hydrocarbyl or heterocarbyl moiety as previously defined wherein one or more hydrogen atoms have been replaced with a chemical group, which does not adversely affect the desired preparation of the modified polysaccharide. Representative of such groups are amino, phosphino, quaternary nitrogen (ammonium), quaternary phosphorous (phosphonium), hydroxyl, amide, alkoxy, mercapto, nitro, alkyl, halo, sulfone, sulfoxide, phosphate, phosphite, carboxylate, carbamate groups and the like.

Variables such as L³-L⁵, R¹′ R², Z, and n used throughout the application are the same variables as previously defined unless stated to the contrary.

### I. Composites

Described herein are composites useful in harvesting a variety of cells and tissue engineering. In one aspect, the composite comprises the reaction product between a macromolecule comprising at least one thiol group and a gold nanoparticle, wherein the gold nanoparticle comprises a cluster of gold atoms, wherein the cluster has a size of 0.5 nm to 250 nm. Each component present in the composite and methods of making and using the composites are described below.

A. Macromolecules

A macromolecule as disclosed herein is any compound having at least one thiol group. These compounds are also referred to herein as “thiolated macromolecules.” The thiol groups present on the macromolecule can be naturally-occurring or incorporated into the macromolecule to produce a chemically-modified macromolecule. In some aspects, the macromolecule can be selected to augment (i.e., increase or decrease) cell adherence to the composites further described below. The number of thiol groups on the macromolecule can vary. In one aspect, the number of thiol groups per macromolecule is from 2 to 4,000, 4 to 1,000, 8 to 250, or 16 to 125.

In one aspect, the macromolecule is a polysaccharide. Any polysaccharide known in the art can be used herein. Examples of polysaccharides include starch, cellulose, glycogen or carboxylated polysaccharides such as alginic acid, pectin, or carboxymethylcellulose. In one aspect, the polysaccharide is a glycosaminoglycan (GAG). A GAG is one molecule with many alternating subunits. For example, HA is (GlcNAc-GlcUA-)x. Other GAGs are sulfated at different sugars. Generically, GAGs are represented by the formula A-B-A-B-A-B, where A is a uronic acid and B is an aminosugar that is either O- or N-sulfated, where the A and B units can be heterogeneous with respect to epimeric content or sulfation.

There are many different types of GAGs, having commonly understood structures, which, for example, are within the disclosed compositions, such as hyaluronic acid, chondroitin sulfate, dermatan, heparan, heparin, dermatan sulfate, and heparan sulfate. Any GAG known in the art can be used in any of the methods described herein. Natural and synthetic polysaccharides such as pullulan, alginic acid, pectin, chitosan, cellulose, or carboxymethylcellulose can also be modified by the methods described herein. Glycosaminoglycans can be purchased from Sigma, and many other biochemical suppliers. Alginic acid, pectin, and carboxymethylcellulose are representative of other carboxylic acid containing polysaccharides useful in the methods described herein. The polysaccharides may also be chemically sulfated to increase their anionic character, a feature important for retaining basic polypeptides in the crosslinked network.

In one aspect, the polysaccharide is hyaluronan (HA), which is the salt of hyaluronic acid. HA is a non-sulfated GAG. Hyaluronan is a well known, naturally occurring, water soluble polysaccharide composed of two alternatively linked sugars, D-glucuronic acid and N-acetylglucosamine. The polymer is hydrophilic and highly viscous in aqueous solution at relatively low solute concentrations. It often occurs naturally as the sodium salt, sodium hyaluronate. Methods of preparing commercially available hyaluronan and salts thereof are well known. Hyaluronan can be purchased from Seikagaku Company, Novozymes Biopolymers, Inc., LifeCore, Inc., Hyalose, Inc., Genzyme, Inc., Pharmacia Inc., Sigma Inc., and many other suppliers. For high molecular weight hyaluronan it is often in the range of 100 to 10,000 disaccharide units. In another aspect, the lower limit of the molecular weight of the hyaluronan is from 1,000 Da, 2,000 Da, 3,000 Da, 4,000 Da, 5,000 Da, 6,000 Da, 7,000 Da, 8,000 Da, 9,000 Da, 10,000 Da, 20,000 Da, 30,000 Da, 40,000 Da, 50,000 Da, 60,000 Da, 70,000 Da, 80,000 Da, 90,000 Da, or 100,000 Da, and the upper limit is 200,000 Da, 300,000 Da, 400,000 Da, 500,000 Da, 600,000 Da, 700,000 Da, 800,000 Da, 900,000 Da, 1,000,000 Da, 2,000,000 Da, 4,000,000 Da, 6,000,000 Da, 8,000,000 Da, or 10,000,000 Da where any of the lower limits can be combined with any of the upper limits

In another aspect, the macromolecule can be gelatin, collagen, cross-linked collagen, or a collagen derivative, such as, succinylated collagen or methylated collagen.

As described above, any of the macromolecules described herein can be chemically modified so that at least one thiol group is present. For example, the procedures and techniques disclosed in International Publication No. WO 2004/037164, which are incorporated by reference in their entirety, can be used to produce thiolated macromolecules with hydrazide groups. In one aspect, the macromolecule has the formula IV

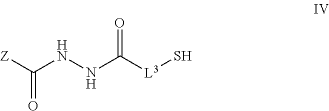

wherein
Z is a residue of a macromolecule; and
L³ is a polyalkylene group, a polyether group, a polyamide group, a polyimino group, an aryl group, a polyester, or a polythioether group.

The macromolecules having the formula IV and methods for producing the same disclosed in U.S. Publication No. 2005/176620, which are incorporated by reference, can be used herein. In one aspect, Z is hyaluronan and L³ is CH₂CH₂ or CH₂CH₂CH₂. In another aspect, Z is hyaluronan or gelatin and L³ is CH₂CH_{2.} These compounds are referred to herein as HA-DTPH and Gelatin-DTPH, respectively.

In another aspect, the macromolecule has the formula V

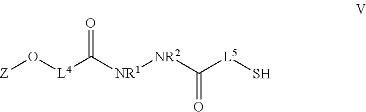

wherein
Z is a residue of a macromolecule; and
R¹ and R² are, independently, hydrogen, a substituted or unsubstituted hydrocarbyl group, a substituted or unsubstituted heterohydrocarbyl group, or a polyether group;
L⁴ and L⁵ are, independently, a substituted or unsubstituted hydrocarbyl group, a substituted or unsubstituted heterohydrocarbyl group, a branched- or straight-chain alkylene group, a polyether group, a polyamide group, a polyimino group, an aryl group, a polyester, a polythioether group, a polysaccharyl group, or a combination thereof.

The macromolecules having the formula V and methods for producing the same disclosed in U.S. Publication No. 2008/025950, which are incorporated by reference, can be used herein. In one aspect of formula V, R¹ and R² are hydrogen. In another aspect of formula V, L⁴ and L⁵ are an alkylene group. Examples of alkylene groups can be denoted by the formula —(CH₂)n-, where n is an integer from 1 to 20, 1 to 15, 1 to 10, 1 to 8, 1 to 6 or 1 to 4. In another aspect, L⁴ is CH₂ and L⁵ is CH₂CH₂. In one aspect, Z in formula V comprises chondroitin, chondroitin sulfate, dermatan, dermatan sulfate, heparin, heparan sulfate, alginic acid, pectin, or hyaluronan. In another aspect of formula V, Z is hyaluronan, R¹ and R² are hydrogen, L⁴ is CH₂, and L⁵ is CH₂CH₂. This compound is referred to herein as CMHA-S, Carbylan™-S, or Glycosil™.

Any of the macromolecules described herein used to make the composites can be the pharmaceutically-acceptable salt or ester thereof. In one aspect, pharmaceutically-acceptable salts are prepared by treating the free acid with an appropriate amount of a pharmaceutically-acceptable base. Representative pharmaceutically-acceptable bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like. In one aspect, the reaction is conducted in water, alone or in combination with an inert, water-miscible organic solvent, at a temperature of from about 0° C. to about 100° C. such as at room temperature. In certain aspects where applicable, the molar ratio of the compounds described herein to base used are chosen to provide the ratio desired for any particular salts. For preparing, for example, the ammonium salts of the free acid starting material, the starting material can be treated with approximately one equivalent of pharmaceutically-acceptable base to yield a neutral salt.

In another aspect, if the compound possesses a basic group, it can be protonated with an acid such as, for example, HCI, HBr, or H₂SO₄, to produce the cationic salt. In one aspect, the reaction of the compound with the acid or base is conducted in water, alone or in combination with an inert, water-miscible organic solvent, at a temperature of from about 0° C. to about 100° C. such as at room temperature. In certain aspects where applicable, the molar ratio of the compounds described herein to base used are chosen to provide the ratio desired for any particular salts. For preparing, for example, the ammonium salts of the free acid starting material, the starting material can be treated with approximately one equivalent of pharmaceutically-acceptable base to yield a neutral salt.

Ester derivatives are typically prepared as precursors to the acid form of the compounds. Generally, these derivatives will be lower alkyl esters such as methyl, ethyl, and the like. Amide derivatives —(CO)NH₂, —(CO)NHR and —(CO)NR₂, where R is an alkyl group defined above, can be prepared by reaction of the carboxylic acid-containing compound with ammonia or a substituted amine.

B. Gold Nanoparticles

The gold nanoparticles useful herein are a cluster of gold atoms. Gold nanoparticles are also referred to as colloidal gold. The gold nanoparticles can be produced using techniques known in the art. In one aspect, the gold nanoparticles can be produced in a liquid by reduction of chloroauric acid (HAuCl₄). After dissolving HAuCl₄, the solution is rapidly stirred while a reducing agent is added, which causes Au³⁺ ions to be reduced to neutral gold atoms. Specific, non-limiting methods for producing gold nanoparticles useful herein are provided in the Examples. The size of the gold nanoparticles can vary. In one aspect, the gold nanoparticles have a particle size ranging from 0.5 nm to 250 nm, 0.5 nm to 200 nm, 0.5 nm to 150 nm, 0.5 nm to 100 nm, 1 nm to 60 nm, 2 nm to 40 nm, or 4 nm to 20 nm.

### II. Preparation of Composites

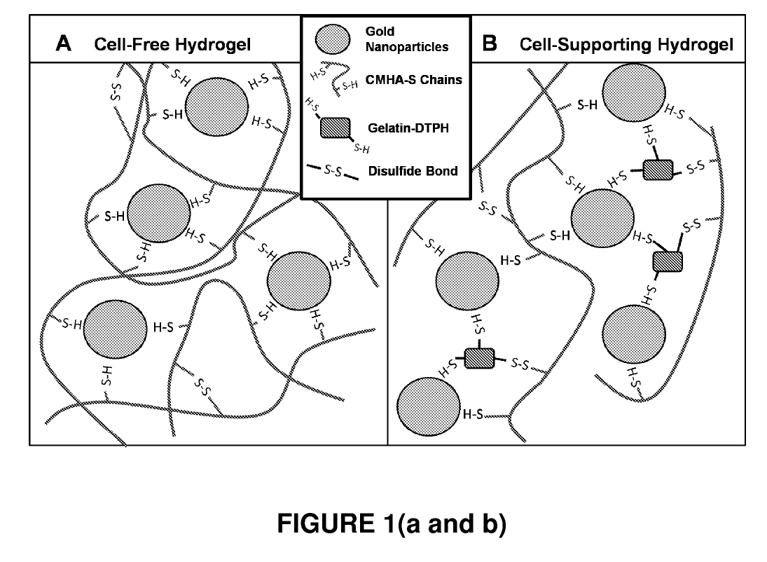

Methods for preparing the composites are also described herein. In one aspect, the method involves crosslinking one or more thiolated macromolecules with a plurality of gold nanoparticles. When any of thiolated macromolecules described herein and gold nanoparticles are mixed with one another, the thiol groups present on thiolated macromolecule can bond with the gold nanoparticles. The mode of bonding can vary depending upon the reaction conditions; however, it is generally accepted that Au-thiolate bonds are relatively strong, covalent interactions, with a bond strength of about 44 kcal/mole. FIG. 1 is a diagram showing the crosslinking strategy used to prepare the composites described herein. Thiols on the CMHA-S chains (as one example of a thiolated macromolecule) form bonds with the surface of the gold nanoparticles to create a network. Importantly, the gold nanoparticles act as nanoscale-multivalent crosslinkers, where the entire surface of the nanoparticle may link to multiple thiol groups. Both inter- and intra-molecular crosslinking can occur between the thiolated macromolecules and gold nanoparticles.

Methods for preparing the composites generally do not require special techniques or handling. In one aspect, the method involves admixing one or more thiolated macromolecules with the gold nanoparticles in water. For example, an aqueous solution of gold nanoparticles can be added to an aqueous solution of one or more thiolated macromolecules to produce the composites described herein. Specific, non-limiting methods for producing the composites are provided in the Examples. The amount of gold nanoparticles used can vary depending upon the selection and amount of thiolated macromolecule used and the end-use of the resulting composite. In one aspect, the weight ratio of thiolated macromolecule to gold nanoparticles is from 5,000:1 to 100:1; 4,000:1 to 100:1; 3,000:1 to 100:1; 2,000:1 to 100:1; 1,000:1 to 100:1; or 1,000:1 to 500:1. Additionally, by varying the size of the gold nanoparticles, the amount of thiolated macromolecule, and the crosslinking time and temperature, it is possible to modify one or more properties of the composite. For example, the degree of crosslinking can influence the viscosity of the composite, which can be important in certain application as discussed below.

### III. Uses

The composites described herein can be used as substrates for harvesting cells. In one aspect, a method for harvesting cells includes:
(a) depositing a parent set of cells on or encapsulated within a composite described herein;
(b) culturing the composite with the deposited cells to promote the growth of the cells;
(c) contacting the composite with a biologically-compatible thiol-containing agent, wherein the thiol-containing agent dissolves the composite and releases the cells from the composite; and
(d) recovering the released cells.

Many types of cells can be harvested (e.g., grown and/or differentiated) using the composites described herein including, but not limited to, stem cells, committed stem cells, differentiated cells, and tumor cells. Examples of stem cells include, but are not limited to, CD34+ stem cells, embryonic stem cells, bone marrow stem cells, placental stem cells, adipose-derived stem cells, liver-derived stem cells, cardiac stem cells, cancer stem cells, neural stem cells, umbilical cord blood stem cells, and induced pluripotent fibroblast stem cells. Other examples of cells used in various embodiments include, but are not limited to, osteoblasts, myoblasts, neuroblasts, fibroblasts, glioblasts, germ cells, hepatocytes, chondrocytes, epithelial cells, cardiovascular cells, keratinocytes, smooth muscle cells, cardiac muscle cells, connective tissue cells, glial cells, epithelial cells, endothelial cells, hormone-secreting cells, cells of the immune system, pancreatic islet cells, and neuronal cells.

Cells useful herein can be cultured in vitro, derived from a natural source, genetically engineered, or produced by any other means. Any natural source of prokaryotic or eukaryotic cells can be used. It is also contemplated that cells can be cultured ex vivo.

Atypical or abnormal cells such as tumor cells can also be used herein. Tumor cells cultured on the composites described herein can provide more accurate representations of the native tumor environment in the body for the assessment of drug treatments. Growth of tumor cells on the substrates described herein can facilitate characterization of biochemical pathways and activities of the tumor, including gene expression, receptor expression, and polypeptide production, in an in vivo-like environment allowing for the development of drugs that specifically target the tumor. Heterogeneous cell populations from patient-derived tumor tissue or stromal tissue can also be cultured in these gels for expansion and recovery or for developing tumors to evaluated candidate anti-cancer agents.

Cells that have been genetically engineered can also be used herein. The engineering involves programming the cell to express one or more genes, repressing the expression of one or more genes, or both. Genetic engineering can involve, for example, adding or removing genetic material to or from a cell, altering existing genetic material, or both. Embodiments in which cells are transfected or otherwise engineered to express a gene can use transiently or permanently transfected genes, or both. Gene sequences may be full or partial length, cloned or naturally occurring.

The cells can be deposited on the surface of the composite using techniques known in the art. Alternatively, the cells can be encapsulated within the composite. For example, a partially gelled composite described herein can be seeded with cells, where the cells can sink into the composite. After the composite gels completely, the seeds are encapsulated by the composite.

After the cells have grown for a sufficient time in the composite, the cells need to be recovered. In general, the recovery of cells from hydrogel composites is not trivial. Degradation of the composite results in the formation of thiolated macromolecules and subsequent release of the cells. The cells can then be recovered using techniques known in the art such as, for example, gentle centrifugation. By using the composites and methods described herein, the harvested cells can be used for research, biomarker identification, production of monoclonal antibodies or other therapeutic proteins, toxicology, drug discovery, or therapeutic purposes. The degradation of the composite can be performed by a number of techniques. In one aspect, the composite can be degraded by contacting the composite with a biologically-compatible thiol-containing agent such as, for example, N-acetyl-L-cysteine, L-cysteine, or glutathione. Not wishing to be bound by theory, the biologically-compatible thiol-containing agent displaces the thiolated macromolecule from the gold nanoparticles, which ultimately dissolves the composite and releases the cells from the composite.

The composites described herein can be formed into a number of different substrates including, but not limited to, a laminate, a gel, a sponge, a film, a mesh, an electrospun nanofiber, a woven mesh, or a non-woven mesh. In one aspect, the composites can be formulated into beads. In this aspect, one or more composites described herein are used alone to manufacture the bead or can be used in combination with other materials. Thus, in this aspect, the composite is incorporated throughout the bead. In certain aspects, it may be desirable to add cells to the mixture used to produce the bead. In this aspect, the cells and composite(s) described herein are incorporated throughout the bead. In other aspects, a bead is coated with one or more composites described herein. Materials useful in making beads include, but are not limited to, polysaccharides (e.g., dextrans), proteins and glycoproteins (e.g., gelatin, thiolated gelatin, and other collagen derivatives), and synthetic polymers (e.g., polystyrenes). Cells may attach and grow on the outer surface and throughout the beads. The beads are generally porous, and cells may attach and grow on the porous inner surfaces of the bead as well as the outer surfaces. Such beads allow for greater surface area on which the cells are grown and further allow for the optimization of growing cells within a cell culture vessel. The cells and beads or the cells attached to the beads may be placed into an incubator or bioreactor and allowed to grow. One skilled in the art would readily know how to culture the cells. As stated above, the cells which are attached to the beads may then be readily dissociated and recovered using the techniques described above.

In other aspects, the composites described herein can be used in tissue engineering. Bioprinting has emerged as an attractive tissue engineering method for building organs. The combination of biocompatible materials and rapid prototyping makes provides a way to address the intricacies needed in viable tissues. One of the hurdles associated with bioprinting is the interfacing between the printing hardware and different types of bio-ink being printed. Standard hydrogels pose design problems because they are either printed as fluid solutions, limiting mechanical properties, or printed as solid hydrogels and broken up upon the extrusion process. The composites described herein address these issues by being mechanically sound and by being able to reversibly crosslink after the printing process. In addition, as discussed above, the composites can be degraded on demand, creating a versatile system for bioprinting.

The composites described herein permit the formation of three-dimensional layered structures or composites. A plurality of cells or cell aggregates can be deposited on composite. This results in the formation of a biological composite, which is the base composite layer. The cells or cell aggregates can be applied to the composite in a predetermined pattern using techniques described below. Multiple biological composites can be applied to the base composite layer to produce a three dimensional structure. The cells or cell aggregates can fuse to one another to produce a tissue or organ. Removal of the composite using the techniques described above results in the isolation of the tissue or organ.

The cells or cell aggregates deposited on the composite are also referred to herein as “bio-ink ” Bio-inks are often used in conjunction with a biopaper, and the subject of this application is the creation of a novel composite that can act as a bio-paper per se, or as a component of a bio-ink That is, the composites described herein are a printable biopaper, or it may be used as a vehicle in the preparation of bio-inks The bio-inks and methods for making the same described in U.S. Published Application No. 2008/0070304 are useful herein, the teachings of which are incorporated by reference in their entirety. In one aspect, the bio-ink is composed of a plurality of cell aggregates, wherein each cell aggregate includes a plurality of living cells, and wherein the cell aggregates are substantially uniform in size and/or shape. The cell aggregates are characterized by the capacity: 1) to be delivered by computer-aided automatic cell dispenser-based deposition or “printing,” and 2) to fuse into, or consolidate to form, self-assembled histological constructs. In certain aspects, the bio-ink is composed of a plurality of cell aggregates that have a narrow size and shape distribution (i.e., are substantially uniform in size and/or shape). By “substantially uniform in shape” it is meant that the spread in uniformity of the aggregates is not more than about 10%. In another aspect, the spread in uniformity of the aggregates is not more than about 5%. The cell aggregates used herein can be of various shapes, such as, for example, a sphere, a cylinder (e.g., with equal height and diameter), rod-like, or cuboidal (i.e., cubes), among others.

Although the exact number of cells per aggregate is not critical, the size of each aggregate (and thus the number of cells per aggregate) is limited by the capacity of nutrients to diffuse to the central cells, and that this number may vary depending on cell type. Cell aggregates may include a minimal number of cells (e.g., two or three cells) per aggregate, or may include many hundreds or thousands of cells per aggregate. Typically, cell aggregates include hundreds to thousands of cells per aggregate. In one aspect, the cell aggregates are from about 100 microns to about 600 microns, or about 250 microns to about 400 microns in size.

Many cell types may be used to form the bio-ink cell aggregates. In general, the choice of cell type will vary depending on the type of three-dimensional construct to be printed. For example, if the bio-ink particles are to be used to print a blood vessel type three dimensional structure, the cell aggregates can include a cell type or types typically found in vascular tissue (e.g., endothelial cells, smooth muscle cells, etc.). In contrast, the composition of the cell aggregates may vary if a different type of construct is to be printed (e.g., intestine, liver, kidney, etc.). One skilled in the art will thus readily be able to choose an appropriate cell type(s) for the aggregates, based on the type of three-dimensional construct to be printed. In addition to the cells described above, non-limiting examples of suitable cell types include contractile or muscle cells (e.g., striated muscle cells and smooth muscle cells), neural cells, connective tissue (including bone, cartilage, cells differentiating into bone forming cells and chondrocytes, and lymph tissues), parenchymal cells, epithelial cells (including endothelial cells that form linings in cavities and vessels or channels, exocrine secretory epithelial cells, epithelial absorptive cells, keratinizing epithelial cells, and extracellular matrix secretion cells), and undifferentiated cells (such as embryonic cells, stem cells, and other precursor cells), among others.

The bio-ink particles may be homocellular aggregates (i.e., “monocolor bio-ink”) or heterocellular aggregates (i.e., “multicolor bio-ink”). “Monocolor bio-ink” includes a plurality of cell aggregates, wherein each cell aggregate includes a plurality of living cells of a single cell type. In contrast, “multicolor bio-ink” includes a plurality of cell aggregates, wherein each individual cell aggregate includes a plurality of living cells of at least two cell types, or at least one cell type and extracellular matrix (ECM) material, as discussed below.

In addition to one or more cell types, the bio-ink aggregates can further be fabricated to contain extracellular matrix (ECM) material in desired amounts. For example, the aggregates may contain various ECM proteins (e.g., collagen, vitronectin, fibronectin, laminin, elastin, and/or proteoglycans). Such ECM material can be naturally secreted by the cells, or alternately, the cells can be genetically manipulated by any suitable method known in the art to vary the expression level of ECM material and/or cell adhesion molecules, such as selectins, integrins, immunoglobulins, and cadherins, among others. In another aspect, either natural ECM material or any synthetic component that imitates ECM material can be incorporated into the aggregates during aggregate formation, as described below. In another aspect, growth factors such as epidermal growth factor, fibroblast growth factors, angiopoetins, platelet derived growth factors, vascular endothelial growth factor, and the like, can be incorporated into the bio-ink or into the bio-paper.

The composites described herein can be used to produce three-dimensional fused aggregates (e.g., tissue or organs). In one aspect, the method involves: (1) depositing a first layer of biological composite as described herein on a substrate; (2) applying one or more layers of additional biological composite on the first layer, wherein each additional layer comprises at least one cell aggregate, the cell aggregate being arranged in a first predetermined pattern; (3) allowing at least one aggregate of said plurality of first cell aggregates to fuse with at least one other aggregate of the plurality of first cell aggregates to form the desired structure; and (4) separating the structure from the composite.

In another aspect, the method involves: (1) depositing a first layer of a composite described herein on a substrate; (2) embedding a plurality of first cell aggregates, each comprising a plurality of first cells, in the composite, the aggregates being arranged in a first predetermined pattern; (3) allowing at least one aggregate of said plurality of first cell aggregates to fuse with at least one other aggregate of the plurality of first cell aggregates to form the desired structure; and (4) separating the structure from the composite. In an additional aspect, one or more layers of composite can be sequentially applied to the first layer, where cell aggregates are applied to each layer prior to addition of the next layer of composite. In the methods described above, the cell aggregates can be dispensed in a predetermined pattern on the composite using any of a variety of printing or dispensing devices as disclosed in U.S. Published Application No. 2008/0070304. The fused aggregate can be released and isolated from the 3-D matrix by degrading the composite using techniques described above for degrading the composite.

It is understood that any given particular aspect of the disclosed compositions and methods can be easily compared to the specific examples and embodiments disclosed herein, including the non-polysaccharide based reagents discussed in the Examples. By performing such a comparison, the relative efficacy of each particular embodiment can be easily determined. Particularly preferred compositions and methods are disclosed in the Examples herein, and it is understood that these compositions and methods, while not necessarily limiting, can be performed with any of the compositions and methods disclosed herein.

### EXAMPLES

The following examples are put forth so as to provide those of ordinary skill in the art with a complete disclosure and description of how the compounds, compositions, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process. Only reasonable and routine experimentation will be required to optimize such process conditions. In addition, additional technical and chemical details, protocols, and publication lists are available on the Glycosan BioSystems website, www.glycosan.com.

### Materials and Methods

Synthesis of 14 nm AuNPs. To 250 mL of water was added 0.17 mL of a 30% wt solution of HAuCl₄ in HCl, and the solution was heated to boiling. Then, 25 mL of a 39 mM sodium citrate solution was added rapidly to the stirred gold solution. The yellow color of AuCl₄⁻ anion disappeared immediately, and after about 1 minute, the solution slowly became violet and then deepened to a wine-red color. The solution was boiled for another 30 minutes and then allowed to cool, and then stirred overnight at room temperature and filtered before use through a 0.45 μm syringe filter (Millipore), resulting in a 10.7 nM solution.

Synthesis of 4 nm AuNPs. Gold nanoparticles were synthesized by adding glutathione to an aqueous solution of HAuCl₄. For a typical preparation of 4 nm particles, 0.5 moles of HAuCl₄ dissolved a 5% (w/v) aqueous solution was at first mixed with 1.25 mmol of glutathione in methanol to give a transparent solution in a 250 mL round flask. A freshly prepared 0.2 M aqueous sodium borohydride solution (25 mL) was then added at a rate of 5 mL per minute under vigorous stirring. The solution turned dark-brown immediately but remained transparent until approximately 13 mL of reductant was added. Further addition of the reductant resulted in a flocculent dark-brown precipitate. After stirring for 1 h, the solvent was removed by decantation after the centrifugation force of 9840 g (10000 rpm) was reached and maintained for 5 minutes. The precipitate was washed twice with a 20% (v/v) water/methanol solution through an ultrasonic redispersion-centrifugation process to remove inorganic or organic impurities. This process was repeated with methanol to remove unbound glutathione. At last, the precipitate was suspended in ethanol and dried by rotary evaporation without a exceeding a temperature of 40 degree, and the residue was dried by vacuum for overnight, giving 170 mg of powder. The powder was resuspended in nanopure water to a final concentration of 10.7 nM, matching that of the 14 nm AuNP solution.

Preparation of hydrogels. Glycosil (Glycosan BioSystems, Inc.), a thiol-modified HA derivative also referred to in this application as CMHA-S, was dissolved in 2× phosphate buffered saline (PBS), to form a 2.5% w/v solution. The solution was adjusted with 1N NaOH to a pH of 7.45. The Glycosil solution was then combined with the AuNP solution in a 15:1 volume ratio. The combined solution was vortexed and drawn into syringes and allowed to crosslink for 24 to 48 hours, affecting the stiffness of the resulting hydrogel. For cell containing hydrogels, CMHA-S was mixed in a 3:1 (w/w) ratio with thiol-modified gelatin to provide for cell attachment and proliferation and then combined with the AuNP solution in a 15:1 volume ratio, and the solutions were pipetted into 24-well plates where they were allowed to crosslink.

Manual extrusion and reversible crosslinking. Initial attempts at printing the hydrogels were performed by hand. After allowing the AuNP-Glycosil solutions to crosslink for 48 h, the gels were extruded through an 18-gauge needle tip. Tubes were created one at a time, and layered to create various geometries.

During preliminary work with these hydrogels, it was observed that gel “pieces” appear to reattach to one another after the extrusion to form a single continuous gel. This property was evaluated qualitatively by prodding the hand-printed gel structures with tweezers and determining visually whether or not the gels would separate from one another from the mechanical force.

After 48 h, the resulting gels could hold their shape, but were soft enough to be able to be extruded through 18-gauge needle tips. The hydrogel tubes that were extruded were not completely uniform, since they partially fragmented while being sheared through the smaller diameter syringe needle. However, the printed tubes maintained a cylindrical form, making it feasible to evaluate using them for further printing of simple structures.

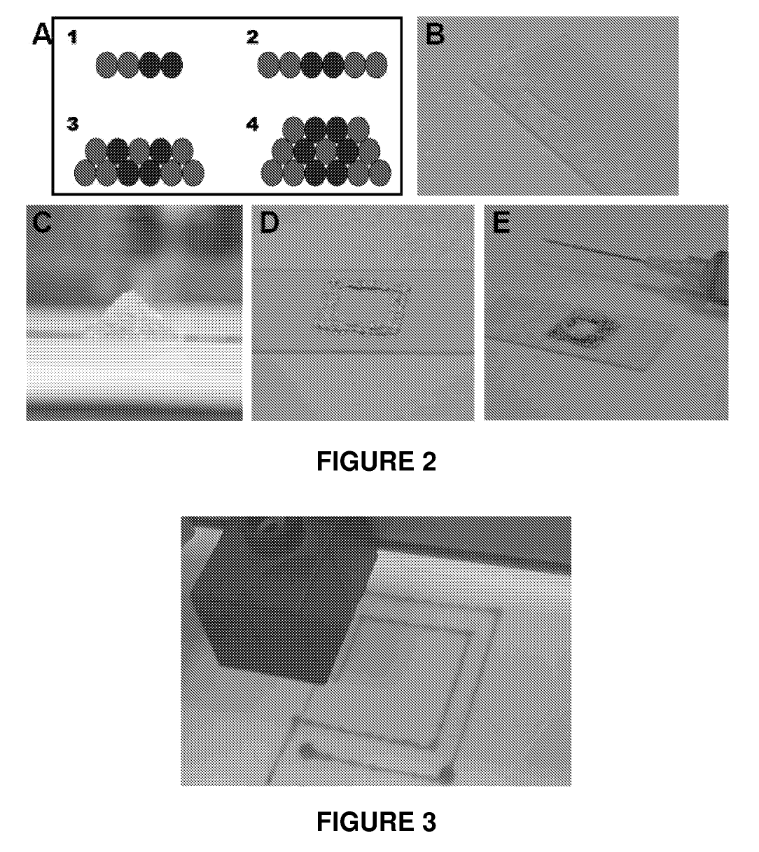

Manual printing was continued by laying down layers of multiple tubes on top of one another (FIG. 2). In this fashion, several simple structures like linear and square stacks of tubes were built. The printed hydrogel tubes were able to support themselves and layers above them, allowing some of the structures to be built as high as 5 layers. These structures could undoubtedly be built higher by extending the width of their bases.

Immediately after printing, the individual extruded hydrogel tubes were distinct, and could be separated with little mechanical force. After 60 min, the individual hydrogel tubes were visible, but could no longer be easily separated. Instead, it appeared that the entire structure was one continuous piece of hydrogel, indicating that the hydrogels had quickly reformed crosslinks with one another upon touch. This phenomenon can be explained by the slow crosslinking speed of these hydrogels and the multivalent, readily exchangeable Au-thiol interactions with the AuNP “crosslinkers.” At the time of printing only a portion of the thiols have formed bonds with the AuNPs, most likely for steric and viscosity reasons. After the first Au-thiol bonds form, the viscosity slows formation of new intermolecular bonds, while the need for the HA-thiol macromolecule to reorient and expose new thiols slows formation of new intramolecular (actually intracomplex) interactions. The similar sizes of the 40 nm AuNPs and thiol-modified HA derivatives results in slow diffusion and thus slow gelation, but also accounts for the reversible crosslinking that can continue among freshly printed hydrogel structural elements.

Mechanically-Driven Printing. Hydrogels were printed using two printing devices. First, a syringe pump-driven printing device was used to see if the gels could be printed through a longer tube, perhaps smoothing out the gel and making it more uniform. Automated printing of the AuNP-Glycosil hydrogel was attempted using a bioprinter from nScrypt, Inc. Hydrogels were prepared as before, and transferred into new syringes designed for use in the bioprinter. The hydrogels were printed under pressure onto microscope slides to assess the compatibility of the hydrogel with the bioprinter.

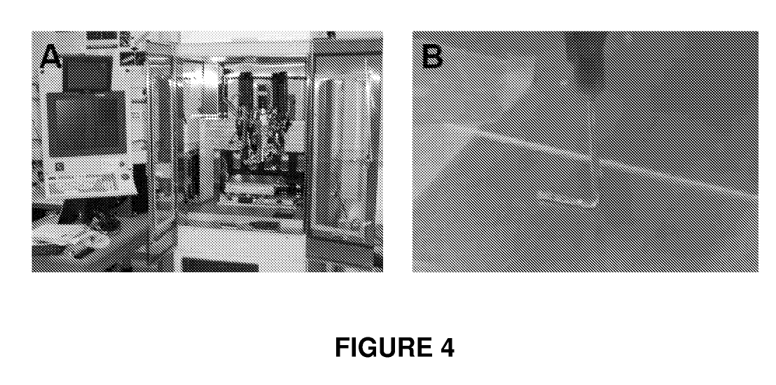

The ability of the gel to be printed varied between formulations, but in general, the materials were able to be extruded onto a stage successfully (FIG. 3). Air bubbles present in the hydrogel from the transfer caused uneven pressure distributions in the syringe, creating some dispensing problems. The hydrogel preparation method will be modified to address these problems. Despite these shortcomings, straight and uniform gel tubes were successfully printed, showing the feasibility of this method (FIG. 4).

Biocompatibility. The Gelin-S (Gelatin-DTPH, Glycosan BioSystems) containing version of the AuNP-Glycosil hydrogel was seeded with 50,000 HepG2 C3A or Int-407 cells per well in 24-well plates. The cells were cultured with Minimum Essential Media Eagle and Basal Media Eagle (Sigma), respectively, both containing 10% fetal bovine serum. Media was changed on day 3. Viability was assessed using LIVE/DEAD (Invitrogen) staining under fluorescent microscopy (n=4). Percent viable cells was determined by # Live Cells/(Total # Cells).

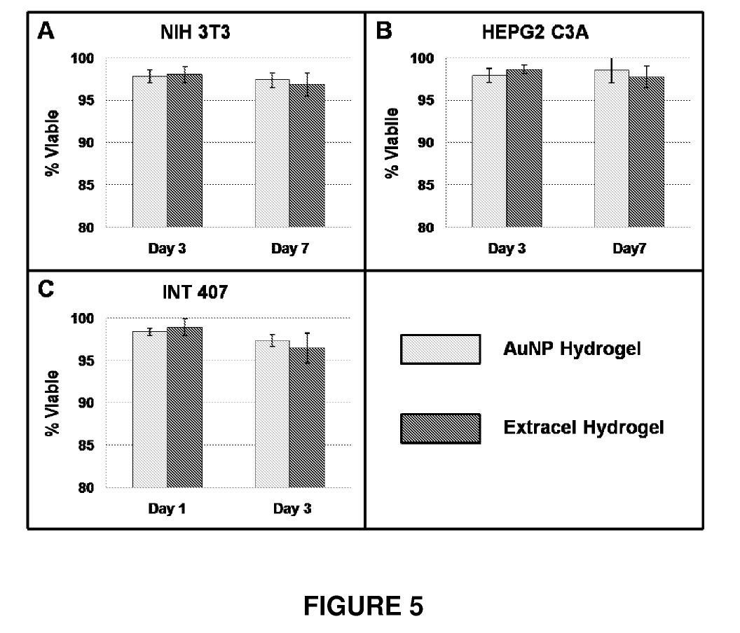

The results showed viability above 95% for all three cell types on AuNP gels, indicating good biocompatibility (FIG. 5). Culture on was performed in parallel on Extracel hydrogels (Glycosan BioSystems, Inc.) as a control. Extracel is a hydrogel in which the same macromolecular thiols, Glycosil and Gelin-S, are crosslinked with poly (ethyleneglycol) diacrylate. Statistics showed no significance between cell viability on different hydrogel types, indicating that the AuNP hydrogels are as biocompatible as Extracel, making them a safe alternative for hydrogel cell culture.

Degradation. An important aspect of a printable hydrogel would be the ability to dissolve and remove the gel on demand. The thiol-gold chemistry allows thiol-containing biocompatible reagents such as cysteine or glutathione in large molar excess to be used to displace the Au-thiol bonds between the thiolated HA chains and the AuNPs. Thus, 25 mM NAcCys solutions in PBS were prepared for the degradation and brought to a pH of 7.4. AuNP-Glycosil hydrogels of 0.2, 0.5, and 1.0 ml volumes were prepared and allowed to crosslink for 48 hours. Each hydrogel was placed in 5 ml of the NAcCys and placed on a rocker in an incubator (37° C.). At 60 min, the 0.2 and 0.5 ml hydrogels were completely dissolved, and at 85 minutes the 1.0 ml hydrogels were completely dissolved. The rate of dissolution can be accelerated by increasing the NAcCys concentration to 50, or even 100, mM, while maintaining cytocompatibility. It is noteworthy that the cell recovery process occurs at ambient to physiological temperatures, and does not require the addition of any enzymes.

At 60 min, the 0.2 and 0.5 ml hydrogels were completely dissolved, and at 85 min, the 1.0 ml hydrogels were completely dissolved. This degradation process can be sped up by increasing the NAcCys concentration to 50, or even 100, mM, while maintaining cytocompatibility if the treatment period is kept under 1 hour. Based on previous research with hydrogels containing degradable crosslinkers, we used these higher concentrations and they did not display ill effects on treated cells.

Rheology. Hydrogels were casted, then tested following a previously published protocol, with the difference that the hydrogels were allowed to cure for 24, 48, 72, or 96 hours on a level surface at 37° C. before testing. Briefly, a 40 mm steel disc was lowered until contacting the gel surface, and G′ was measured using a shear stress sweep test ranging from 0.6 to 20 Pa at an oscillation frequency of 1 Hz applied by the rheometer.

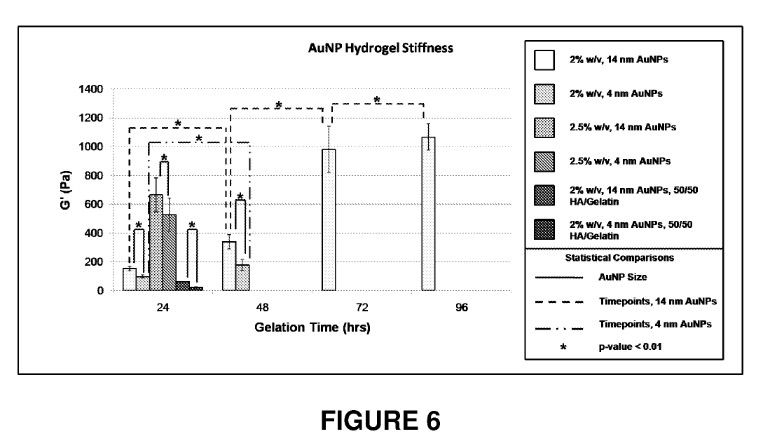

Rheological data from shear stress sweep tests shows that G′, or the storage modulus, which indicates stiffness, is dependent on several factors, but most importantly, G′ is time dependent (FIG. 6). The 48-hour cured hydrogels were much stiffer than the 24-hour cured hydrogels. At 72 and 96 hours the hydrogels' stiffness was even higher and had reached a plateau at ˜1 kPa, indicating complete crosslinking. At 24 hours a 2% hydrogel has a G′ of approximately 200 Pa and is still extrudable through a syringe. At 48, 72, and 96 hours gelation, the hydrogels required too much force to be extruded. This additional crosslinking and stiffening does however, help to stabilize and strengthen the gel during culture. Based on the rheological results and our experimentation with printing formulations, the remaining work was performed with hydrogels crosslinked with 14 nm AuNPs only, hence the lack of data for the 4 nm AuNP gels after this point.

Printing a Tubular Tissue Construct. Using the 2-Syringe Model 1 Fab@Home printing machine (NextFab), tubular constructs were built. Two formulations for hydrogels were prepared for printing. Cell-free and cell-containing hydrogels were prepared as described above. All solutions were adjusted to a pH of 7.4 (1M NaOH) and were sterile filtered through 0.45 μm syringe filters (Millex). The cell containing hydrogel solution was used to encapsulate NIH 3T3s at a density of 25 million cells/ml. Cells were previously cultured to 90% confluency on tissue culture plastic, treated with accutase to detach them from the substrate, counted, split, and centrifuged into a cell pellet. Both hydrogels were drawn into several 10 ml syringes compatible with the printer and placed in a 37° C. incubator for 24 hours. The cell containing syringes were rotated at 10 rpm along their longitudinal axes parallel to the floor, to keep cells suspended while the solution stiffened.

To print a cellular structure a vertical ring-stacking protocol was used. The hydrogels were printed out of the syringes through a 0.25 mm tip. One layer was printed by first laying down cell-free hydrogel in disc-like shape that was 3-5 mm in diameter. Then a ring of cell-containing hydrogel was laid down around the disc that was 1-2 mm thick. Finally, an additional ring of cell-free hydrogel was laid down around the first ring. This process was repeated, building up a tube of cellularized hydrogel contained within cell-free hydrogel. The resulting hydrogel and cellular structures were allowed to sit for 60 minutes in the incubator without additional media in order to allow each printed piece to recrosslink to adjacent pieces. Media was then added to the dishes, and the constructs placed in culture to allow continued hydrogel and tissue fusion to occur.

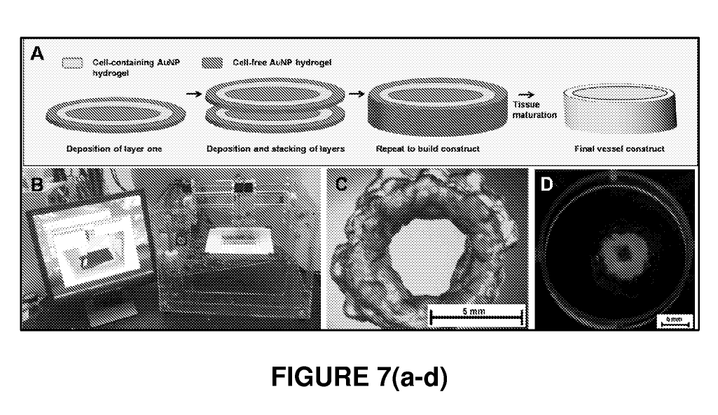

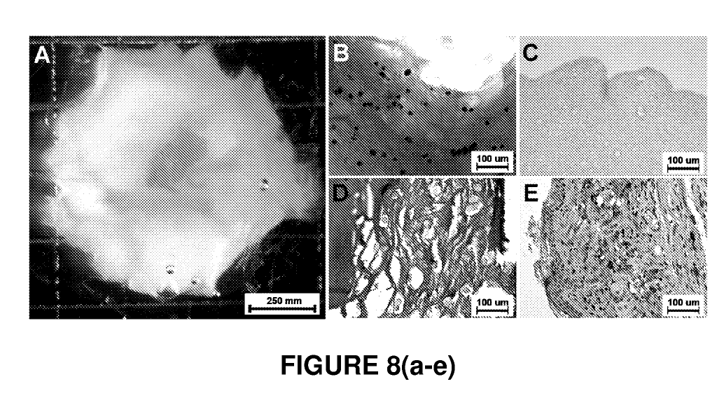

The Fab@Home Model 1 2 syringe printer (NextFab) was used to print hydrogels with and without NIH 3T3 cells (25 million cells/ml) into tubular tissue constructs with a stacked ring printing approach (FIGS. 7A and 7B). Hydrogels made from CMHA-S chains and gold nanoparticles did not contain NIH3T3 cells, and hydrogels made from CMHA-S chains, gold nanoparticles, and gelatin-DTPH contained NIH 3T3 cells. Constructs were 0.8 to 1.0 cm in diameter and were printed from 1 to 2 cm tall using a central core and outer ring of cell free hydrogel as supports, as well as cell migratory barriers to preserve the ring geometry during tissue culture. After printing and 60 minutes of incubation, each construct appeared and felt like all the contained pieces had fused. Surfaces that had been uneven and irregular were now smooth, and the cellular rings were visible within the hydrogels under visible light and with increased contrast under 365 NM UV light using a HA-Bodipy fluorescent dye supplemented to the cell-containing hydrogel (FIGS. 8C and 8D). Over the 4 week culture period, the cellular rings became noticeably more opaque, as the cells proliferated and secreted their own extracellular matrix. At the end of culture, all constructs had retained, if not gained, mechanical properties and were easy to handle for histology protocols.

Histology and Immunohistochemistry. Masson Trichrome was performed using a standard kit (Sigma) and procollagen IHC was performed using a two antibody and DAB substrate protocol (Vector Laboratories).

After 4 weeks of culture, media was aspirated and constructs were fixed in 4% paraformaldehyde in 1× PBS for 4 hours. Samples were then dehydrated with graded ethanol washes, followed by Citrisolv (Fisher Scientific). Samples were paraffin embedded and sectioned at 4 μM. Sections were then stained with Masson Trichrome and H&E for histology. Masson Trichrome staining was accomplished utilizing a standard staining platform kit (Sigma) and slides were imaged under light microscopy for the presence of collagen.

For IHC, all incubations were carried out at room temperature unless otherwise stated. Slides were deparaffinized and hydrated through Citrisolv and graded ethanol washes. Endogenous peroxidase activity was blocked with 1% hydrogen peroxide solution in 1× phosphate-buffered saline solution with 0.1% Tween-20 (PBT) for 20 min. Antigen retrieval was performed on all slides and achieved with microwaving in 1% antigen unmasking solution (Vector Laboratories) for 20 min, then left at room temperature for 30 minutes. IHC was performed using the Vectastain Elite ABC peroxidase kit (Vector Laboratories) according to the manufacturer's protocol. Briefly, non-specific antibody binding was minimized by incubating sections for 90 min in diluted normal blocking serum. Sections were incubated overnight at 4° C. in a humidified chamber with primary anti-procollagen antibodies at a 1:500 dilution. Following overnight incubation, slides were washed in PBT for 9 min. Sections were then incubated for 90 min with biotinylated secondary antibody solution diluted to 5 μg/ml in PBT, followed by Vectastain Elite ABC Reagent (Vector) diluted in PBT for 30 min. Between incubations, sections were washed for 9 min in PBT. Visualization of immunoreactivity was achieved by incubating sections in the DAB peroxidase substrate kit (Vector Laboratories) for 1-2 min. The sections were washed in double distilled H₂O, counterstained with hematoxylin, dehydrated, and cover slipped. Positive control slides of previously sectioned epidermal and dermal tissue were used for comparison. Negative controls were set up at the same time as the primary antibody incubations and included incubation with PBT, in place of the primary antibody. No immunoreactivity was observed in these negative control sections.

After allowing sufficient time for the cells to grow, the cells were analyzed using the methods described above. The increase in opaqueness suggested an increased cell density and production of new ECM by the cells in the construct (FIG. 8A). Masson Trichrome staining showed cells immersed in sheets of collagen fibrils (FIG. 8B). It is important to note that in sectioned areas that were non-cellularized, hydrogel substance was still evident but a lack of collagen was observed. This is evident by the contrast seen between the lumen where no stain was seen and construct walls which stained strongly for collagen. The negative control failed to stain positive for collagen, despite containing gelatin, ensuring that collagen was indeed present in the cellularized construct, and not a false positive (FIG. 8C). As an additional verification of collagen formation, immunohistochemisty (IHC) staining for procollagen, collagen's intracellular precursor, stained positive, indicating that the cells in the constructs were indeed actively producing collagen (FIG. 8D). The positive control tissues showed similar specific staining for procollagen validating our protocol (FIG. 8E). The presence of collagen suggests that the cells reorganized their environment, secreting collagen, and possibly additional ECM components, as they matured into viable tissue during culture.

Throughout this application, various publications are referenced. The disclosures of these publications in their entireties are hereby incorporated by reference into this application in order to more fully describe the compounds, compositions and methods described herein.

Various modifications and variations can be made to the compounds, compositions and methods described herein. Other aspects of the compounds, compositions and methods described herein will be apparent from consideration of the specification and practice of the compounds, compositions and methods disclosed herein. It is intended that the specification and examples be considered as exemplary.

1. A composite comprising the reaction product between a macromolecule comprising at least one thiol group and a gold nanoparticle, wherein the gold nanoparticle comprises a cluster of gold atoms, wherein the cluster has a size of 0.5 nm to 250 nm.

2. The composite of claim 1, wherein the macromolecule comprises a chemically-modified polysaccharide or glycosaminoglycan, wherein the macromolecule naturally comprises at least one thiol group or has been chemically modified to include at least one thiol group.

3. The composite of claim 1, wherein the macromolecule comprises a chemically-modified polysaccharide derived from hyaluronic acid, chondroitin sulfate, dermatan, heparan, heparin, dermatan sulfate, heparan sulfate, alginic acid, pectin, chitosan, or carboxymethylcellulose.

4. The composite of claim 1, wherein the macromolecule comprises a thiol-containing chemically-modified hyaluronan, a thiol-modified gelatin, or a combination thereof.

5. The composite of claim 1, wherein the macromolecule comprises the formula IV 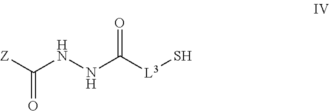
wherein
Z is a residue of a macromolecule; and
L³ is a polyalkylene group, a polyether group, a polyamide group, a polyimino group, an aryl group, a polyester, or a polythioether group.

6. The composite of claim 5, wherein Z comprises chondroitin, chondroitin sulfate, dermatan, dermatan sulfate, heparin, heparan sulfate, alginic acid, pectin, or hyaluronan.

7. The composite of claim 5, wherein L³ is a polyalkylene group having the formula (CH₂)ₙ, where n is from 1 to 10.

8. The composite of claim 5, wherein Z is a residue of hyaluronan and L³ is CH₂CH₂.

9. The composite of claim 1, wherein the macromolecule comprises the formula V 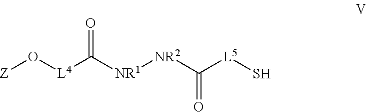
wherein
Z comprises a residue of a macromolecule; and
R¹ and R² comprise, independently, hydrogen, a substituted or unsubstituted hydrocarbyl group, a substituted or unsubstituted heterohydrocarbyl group, or a polyether group;
L⁴ and L⁵ comprise, independently, a substituted or unsubstituted hydrocarbyl group, a substituted or unsubstituted heterohydrocarbyl group, a branched- or straight-chain alkylene group, a polyether group, a polyamide group, a polyimino group, an aryl group, a polyester, a polythioether group, a polysaccharyl group, or a combination thereof.

10. The composite of claim 9, wherein R¹ and R² are hydrogen.

11. The composite of claim 9, wherein L⁴ and L⁵ are an alkylene group.

12. The composite of claim 9, wherein L⁴ is CH₂ and L⁵ is CH₂CH₂.

13. The composite of claim 9, wherein Z comprises chondroitin, chondroitin sulfate, dermatan, dermatan sulfate, heparin, heparan sulfate, alginic acid, pectin, or hyaluronan.

14. The composite of claim 9, wherein Z is hyaluronan, R¹ and R² are hydrogen, L⁴ is CH₂, and L⁵ is CH₂CH₂.

15. The composite of claim 1, wherein the gold nanoparticle comprises an average particle size from 2 to 60 nm.

16. The composite of claim 1, wherein the composite comprises a first thiolated macromolecule and a second thiolated macromolecule, wherein the first thiolated macromolecule and the second thiolated macromolecule are different.

17. The composite of claim 16, wherein the first thiolated macromolecule is CMHA-S and the second thiolated macromolecule is gelatin-DTPH.

18. A method for making the composite of claim 1, wherein the method comprises crosslinking one or more thiolated macromolecules with a plurality of gold nanoparticles.

19. The method of claim 18, wherein the method comprises admixing one or more thiolated macromolecules with the gold nanoparticles in water.

20. The method of claim 18, wherein the weight ratio of thiolated macromolecule to gold nanoparticles is from 5000:1 to 100:1.

21. A composite produced by the method of claim 18.

22. A biological composite of claim 1 comprising a bio-ink.

23. The composite of claim 22, wherein the bio-ink comprises a plurality of cells or cell aggregates, and wherein the cells or cell aggregates are essentially homogeneous or heterogeneous in cell type.

24. The composite of claim 23, wherein the cells or cell aggregates comprise stem cells, osteoblasts, myoblasts, neuroblasts, fibroblasts, glioblasts, germ cells, hepatocytes, chondrocytes, epithelial cells, cardiovascular cells, keratinocytes, smooth muscle cells, cardiac muscle cells, connective tissue cells, glial cells, epithelial cells, endothelial cells, hormone-secreting cells, cells of the immune system, pancreatic islet cells, or neuronal cells.

25. A method for harvesting cells comprising
(a) depositing a parent set of cells on and/or encapsulated within a composite of claim 1;
(b) culturing the composite with the deposited cells to promote the growth of the cells;
(c) contacting the composite with a biologically-compatible thiol-containing agent, wherein the cells are released from the composite; and
(d) recovering the released cells.

26. The method of claim 25, wherein the cells are essentially homogeneous or heterogeneous in cell type.

27. The method of claim 25, wherein the cells comprise stem cells, osteoblasts, myoblasts, neuroblasts, fibroblasts, glioblasts, germ cells, hepatocytes, chondrocytes, epithelial cells, cardiovascular cells, keratinocytes, smooth muscle cells, cardiac muscle cells, connective tissue cells, glial cells, epithelial cells, endothelial cells, hormone-secreting cells, cells of the immune system, pancreatic islet cells, or neuronal cells.

28. The method of claim 25, wherein the agent comprises N-acetyl-L-cysteine, L-cysteine, or glutathione.

29. Cells produced by the method of claim 25.

30. The use of the cells produced by the method of claim 25 for research, biomarker identification, production of monoclonal antibodies or other therapeutic proteins, toxicology, drug discovery, or therapeutic purposes.

31. A three-dimensional layered structure comprising a plurality of biological composites of claim 22, wherein the biological composites are layered on top of one another.

32. The structure of claim 31, wherein the bio-ink embedded in each layer of the composite is deposited on the composite in a predetermined pattern.

33. The structure of claim 31, wherein the bio-ink in each layer of composite is the same.

34. A method of producing a fused aggregate forming a desired three-dimensional structure, the method comprising: (1) depositing a first layer of biological composite of 22 on a substrate; (2) applying one or more layers of additional biological composite on the first layer, wherein each additional layer comprises at least one cell aggregate, the cell aggregate being arranged in a first predetermined pattern; (3) allowing at least one aggregate of said plurality of first cell aggregates to fuse with at least one other aggregate of the plurality of first cell aggregates to form the desired structure; and (4) separating the structure from the composite.

35. A method of producing a fused aggregate forming a desired three-dimensional structure, the method comprising: (1) depositing a first layer of composite of claim 1 on a substrate; (2) embedding a plurality of first cell aggregates, each comprising a plurality of first cells, in the composite, the aggregates being arranged in a first predetermined pattern; (3) allowing at least one aggregate of said plurality of first cell aggregates to fuse with at least one other aggregate of the plurality of first cell aggregates to form the desired structure; and (4) separating the structure from the composite.

36. The method of claim 35, wherein the method further comprises depositing a second layer of a composite comprising the reaction product between a macromolecule comprising at least one thiol group and a gold nanoparticle, wherein the gold nanoparticle comprises a cluster of gold atoms, wherein the cluster has a size of 0.5 nm to 250 nm on the first layer; and embedding a second plurality of cell aggregates in the second layer, the second plurality of cell aggregates comprising a plurality of second cells, the second plurality of cell aggregates being arranged in a second predetermined pattern, and allowing at least one cell aggregate in the first plurality of cell aggregates to fuse with at least one cell aggregate in the second plurality of cell aggregates.

37. A tissue or organ produced by the method of claim 34.

38. A tissue or organ produced by the method of claim 35.
